(19) United States
(12) Patent Application Publication
(10) Pub. No.: US 2011/0124604 A1
(43) Pub. Date: May 26, 2011

(54) PHOSPHOLIPID RECEPTORS AS TARGETS FOR ENHANCING DRUG PERMEABILITY TO SELECTED TISSUES

(76) Inventors: Donald Miller, Winnipeg (CA); Myron Toews, Omaha, NE (US); Sanjat Savant, Concord, CA (US); Bill Mayhan, Omaha, NE (US)

(21) Appl. No.: 12/446,284

(22) PCT Filed: Oct. 22, 2007
(86) PCT No.: PCT/CA2007/001868

§ 371 Date: Jan. 22, 2011

Related U.S. Application Data
(60) Provisional application No. 60/853,288, filed on Oct. 20, 2006.

(51) Int. Cl. A61K 31/661; A61P 25/00

(52) U.S. Cl. 514/114; 514/120

(57) ABSTRACT

A method for enhancing drug delivery to the brain in neurological disorders using lysophosphatidic acid (LPA) and sphingosine I phosphate (S1P) is herein described. Specifically, the permeability properties of LPA and S1P allow for a highly controlled and transient disruption of blood-brain barrier permeability. Thus these phospholipids can be used for delivering a wide variety of therapeutic, prophylactic or diagnostic agents to the brain.

### PRIOR APPLICATION INFORMATION

The instant application claims the benefit of U.S. Provisional Application 60/853,288, filed Oct. 20, 2006.

### STATEMENT OF GOVERNMENT INTERESTS

This invention was made with government support under the U.S. National Institutes of Health grant number R01-CA 93558. The United States Government has certain rights in the invention.

### BACKGROUND OF THE INVENTION

The effects of lysophosphatidic acid (LPA) and sphingosine. I phosphate (S1P) on endothelial cell permeability have been published by various groups. However, studies to date by various groups reveal a contradictory effect of LPA on endothelial barrier. Studies in human umbilical vein endothelial cells, HUVEC (Padden et al, 1997) and brain endothelial cells (Van Hinsbergh et al., 2000) reported an increase in permeability following LPA exposure. The increased permeability was correlated with an increase in actin stress fiber formation, focal contact formation, and Rho and Rho kinase activity, but no obvious changes were apparent in localization of adhesion molecules at the endothelial cell-cell junctions. Studies by Galla at al. (2003) showed that LPA induced a rapid breakdown of the TEER in porcine brain microvessel endothelial cells (PBMEC), indicative of an increase in permeability in these cells. In contrast, LPA decreased endothelial permeability and increased actin stress fiber formation in HUVEC-derived endothelial cell line EA.hy926 and BPAE cells. Endothelial permeability of BPAEs was decreased by a factor present in platelet “releasate” (Haselton at al., 1998). This factor was bound to albumin, extracted with methanol and had an enzyme degradation profile consistent with LPA. Thus while the overriding reports would appear to indicate LPA increases endothelial cell permeability, there are studies reporting a decrease in endothelial cell permeability following LPA exposure. Such discrepancies in the literature with regard to LPA-induced effects on endothelial permeability may be attributable to different LPA receptors and signaling pathways in the various endothelial cell preparations.

In contrast, activation of plasma membrane sphingosine 1-phosphate (S1P) receptors has only been reported to decrease permeability. Decreased permeability in several endothelial cell types in response to S1P is dependent upon activation of Rho and Gi through the Edg1 and Edg3 receptors for S1P. Sphingosine 1-phosphate acting through G, activated Rae and enhanced the cortical cytoskeleton leading to decreased permeability. Furthermore, S1P stimulation of endothelial cells induces VE-cadherin assembly, which plays an important role in regulating solute flux across the endothelial monolayer, Panetti et al. (2002) postulated that both S1P and LPA may decrease endothelial permeability and contribute to the maintenance of the vascular endothelial barrier to protect against tissue edema and hemorrhage. Studies by Garcia and colleagues (2003) further affirm the potential role of S1P in maintaining vascular integrity during inflammatory.

### SUMMARY OF THE INVENTION

According to a first aspect of the invention, there is provided a method of modulating permeability of the blood brain barrier comprising:

administering to an individual in need of such treatment an effective amount of lysophosphatidic acid (LPA) or sphingosine. I phosphate (S1P).

According to a second aspect of the invention, there is provided a method of administering a neural agent to an individual in need of such treatment comprising coadministering an effective amount of the neural agent and an effective amount of LPA to said individual; and following a therapeutically suitable interval, administering an effective amount of S1P to said individual.

According to a third aspect of the invention, there is provided a method of counteracting a disrupted blood brain barrier in an individual in need of such treatment comprising administering to said individual an effective amount of

sphingosine 1-phosphate (S1P).

### BRIEF DESCRIPTION OF THE DRAWINGS

FIG. 1. Permeability effect of LPA (0.1, 1, and 10 uM) on confluent bovine brain microvascular endothelial cell (BBMEC monolayer). LPA effects on BBMEC permeability (enhanced flux of the fluorescent marker FDX-3000) were concentration-dependent effect and apparent as early as 15 min, the earliest time point tested.

FIG. 2. In situ perfusion of rat blood-brain barrier vessels with 10 uM LPA, beginning at 10 minutes above, caused a near}y instant 4-fold increase in blood-brain barrier permeability measured using a “cranial window” model. At about 24 minutes above, the LPA perfusion was stopped, and blood-brain barrier permeability returned to baseline levels extremely rapidly. Fluorescein-labelled dextran of MW 10 kD was used to study permeability in this model. This is the first in vivo study to report LPA effects on permeability of blood-brain barrier, to the best of our knowledge. These live animal data support the likely feasibility of our proposed use of LPA as a transient blood-brain barrier opener.

FIG. 3: Permeability effect of LPA and S1P on confluent BBMEC monolayer. S1P alone was able to enhance barrier function, i.e. to decrease permeability. In addition, S1P was able to completely overcome the increase in permeability induced by LPA. Thus we propose that the opposing effects of LPA and S1P could be used to effectively. increase, decrease, or “clamp” permeabilty at desired levels for tight control of drug delivery to the brain across this cell barrier.

FIG. 4. Permeability effect of various S1P concentrations (0.1, 1, and 10 uM) on confluent BBMEC monolayer. Data are represented as mean+SEM. The experiment was carried at 37° C. (n=3)

FIG. 5. Permeability effect of LPA (10 uM) and S1P (10 uM) on confluent BBMEC monolayer (Basolateral to apical). Data are represented as mean+SEM. The experiment was carried at 37° C. (n=3)

FIG. 6. Polar Permeability effect of S1P (10 uM) on confluent BBMEC monolayer. Data are represented as mean+SEM. The experiment was carried at 37° C. n=3)

FIG. 7. Polar Permeability effect of LPA (10 uM) on BBMEC monolayer. Data are represented as mean±SEM. The experiment was carried at 37° C. (n=3)

FIG. 8. Permeability effects of S1P and horse serum (10%) on confluent BBMEC monolayer. The experiment was carried at 37° C. (n=3)

FIG. 9. Permeability effect of S1P (10 uM) and ethanol (50 mM) on BBMEC monolayer. Data are represented as mean±SEM. The experiment was carried at 37° C. (n=3)

FIG. 10. Permeability effect of LPA in mice. LPA 1 mg/kg was injected along with 4 μCi of 3H-methotrexate via tail vein. (n=5)

FIG. 11. Bar graph showing 20 fold increase in ³H-methotrexate uptake in mouse brain when LPA is present. Adult female Balb/c mice were administered radiolabeled methotrexate via bolus tail vein injection. The radiolabeled methotrexate was given either alone, or in the presence of 1.0 mg/kg LPA. Fifteen minutes after injection of radiolabeled methotrexate, the mice were sacrificed and blood and tissue (brain, lung, kidney and liver) samples removed. The brain was homogenized and the cerebral microvascular endothelial cells were separated from the rest of the brain via centrifugation in 13% dextran solution at 7500×g for 15 minutes. Samples of the brain parachyma were removed for determination of radioactivity along with other tissue homogenates. Under normal conditions less than 1% of the injected dose of radiolabeled methotrexate was delivered to the brain. However, in the presence of LPA, an approximately 20-fold enhancement of radiolabeled methotrexate delivery to the brain was observed. A much lower (approximately 3-fold increase) in methotrexate was observed in the lung following LPA administration, and no significant increases in radiolabeled methotrexate was observed in kidney or liver. These studies demonstrate in a conscious animal, that LPA can be used to increase the delivery of the chemotherapeutic agent methotrexate to the brain. As most anticancer agents do not penetrate the blood-brain barrier in therapeutically relevant amounts, these data demonstrate that activation of LPA receptors on the brain microvessel endothelial cells can be used to increase drug delivery to the brain.

FIG. 12. Adult female Balb/c mice were anesthetized with ketamine xylazine cocktail, and given a bolus i.v. (tail vein) injection of the near infrared fluorescent dye, rhodamine 800 (3.2 μmol/kg) either alone or in the presence of LPA (1.0 mg/kg) or the combination of LPA and S1P (0.1 mg/kg). Mice were sacrificed at 15 minutes following injection of rhodamine 800 and following perfusion with formalin through cardiac puncture, the brain was removed and sliced (3 mm) coronally. The appearance of rhodamine 800 was visualized using an Odyssey near infrared imaging system. Under normal (control) conditions, very little rhodamine 800 dye enters into the brain. The rhodamine 800 is confined primarily to the cerebral spinal fluid present in the ventricles (the small bright regions observed in the various brain slices). However, substantially greater rhodamine 800 was delivered to the brain in the LPA treated mouse. This is evident by the intense bright fluorescence of the brain slices in the LPA treated mouse. Equally important, is that administration of LPA and S1P together prevents the increases in rhodamine 800 brain distribution observed with LPA alone. This suggests that not only can LPA be used to increase the delivery of therapeutic agents or diagnostic markers to the brain, but that S1P can be used to restore the restrictive nature of the blood-brain barrier.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

An important problem in the treatment of neurological disorders is posed by the need to deliver therapeutic agents to the brain. Described herein is a method for enhancing drug delivery to the brain in neurological disorders. Specifically, the permeability properties of lysophosphatidic acid (LPA) and sphingosine I phosphate (S1P) allow for a highly controlled and transient disruption of blood-brain barrier permeability. Thus these phospholipids can be used for delivering a wide variety of therapeutic, prophylactic or diagnostic agents to the brain.

Accordingly, effective amounts of LPA and/or S1P can be used to regulate or modulate permeability of brain endothelial cells and/or the blood brain barrier. Specifically, it is of note that LPA will increase blood brain barrier permeability compared to an untreated control while S1P will decrease blood brain barrier permeability compared to an untreated control.

As discussed below, LPA and S1P can be coadministered with neural agents. For example, LPA can be coadministered with a neural agent that typically has poor delivery to the brain so that when the neural agent is coadministered to an individual in need of such treatment with LPA, the delivery of the neural agent to the brain of the individual is much higher that is the concentration of the agent in the brain is much higher than if the neural agent was administered alone.

Similarly, S1P can be coadministered with a neural toxic agent, that is, an agent which has potential neural toxicity. In these embodiments, the agent coadministered with S1P has lesser neural toxicity than if the neural toxic agent was administered alone.

In other embodiments, there is provided a method for manufacturing a pharmaceutical composition comprising admixing LPA and a neural agent.

In yet other embodiments, there is provided a method for manufacturing a pharmaceutical composition comprising admixing S1P and a neural toxic agent.

In other embodiments, there is provided a pharmaceutical composition comprising LPA and a neural agent.

In yet other embodiments, there is provided a pharmaceutical composition comprising S1P and a neural toxic agent.

In other embodiments, there is provided a method of administering a neural agent to an individual in need of such treatment comprising coadministering an effective amount of the neural agent and an effective amount of LPA to said individual; and following a therapeutically suitable interval, administering an effective amount of S1P to said individual. As will be appreciated by one of skill in the art, in these embodiments, LPA and S1P are used in series so that the magnitude and the duration of the disruption to the blood brain barrier is controlled.

As will be appreciated by one of skill in the art, in these embodiments, LPA is used to increase the permeability of the blood brain barrier so that more of the neural agent is delivered to the brain more quickly. LPA is then added after a sufficient or suitable interval to reduce the permeability of the blood brain barrier. It is of note that the length of such an interval may be easily determined through routine experimentation by one of skill in the art and may depend on several factors, for example, but by no means limited to the neural agent; the age, weight and condition of the individual; and the blood brain barrier permeability of other agents. That is, in some embodiments, the interval may not be based on the condition of the patient or on the agent itself but rather on the presence of other potentially harmful factors which may cross the blood brain barrier.

For example, as discussed below, LPA causes a rapid and reversible increase in blood-barrier permeability. Accordingly, LPA can be co-administered to an individual in need of such treatment with a neural therapy agent, for example, a pharmaceutical agent, a gene therapy agent or the like. Examples of such neural therapy agents include but are by no means limited to various anticancer agents including but by no means limited to, methotrexate, paclitaxel, doxorubicin, topotecan, imatinib mesylate, and cisplatinum; antiviral agents such as acyclovir, zidovudine, and HIV protease inhibitors, for example, saquinavir, nelfinavir, ritonavir, amprenavir, and indinavir; and neuroprotective agents such as nerve growth factor, insulin growth factor erythropoietin, apolipoprotein E and their respective analogs. The present invention can also be used to increase the delivery of gene therapy to the brain for CNS based illnesses such as Parkinson's disease, Huntington's Disease and other degenerative diseases. In addition, the use of this invention to increase delivery of therapeutic proteins to the brain would be desired in the treatment of various lysosomal storage diseases, such as Sanfilippo A, where inherited mutations result in the inability to properly metabolize and process glycoproteins. In these cases enzyme replacement therapy restores normal function, but requires sufficient delivery of enzyme to the brain, which is difficult due to the presence of the blood-brain barrier. The present invention also has applications in the delivery of diagnostic agents to the brain for imaging disease processes. This could include diagnostic antibodies used to identify tumor regions in the brain, or disease processes such as beta-amyloid plagues. Such diagnostic agents could be visualized in the brain using magnetic resonance imaging, or near infrared detection. While an ‘individual in need of such treatment’ will be readily understood by one of skill in the art, it is noted that such individuals will include but are by no means limited to individuals being administered an effective amount of a neural therapy agent as discussed above, in some embodiments, the invention is used for the acute treatment of a CNS related problem, such as a brain tumor, a brain infection, brain trauma. In other embodiments, the BBB could be opened as described above and a nanoparticle or other drug carrier that was loaded with drug is administered as sustained or controlled release from the nanoparticle or microcapsule, thereby delivering these particles, and then allowing the particles to slowly release drug over extended periods of time.

While not wishing to be bound to a particular theory or hypothesis, the inventors believe that the effect of LPA on the blood brain barrier is transient and is both time- and dosage-related, in some embodiments, lasting less than 4 hours, less than 2 hours, less than 1 hour, less than 30 minutes or less than 20 minutes. As will be appreciated by one of skill in the art, the exact time will of course depend on the size, age, weight and condition of the individual as well as other factors including the specific preparation of LPA, the amount administered and the like.

Accordingly, it is noted that the instant invention represents a means for controlling the time that the blood brain barrier is open and also provides means for closing the blood brain barrier in a controlled manner. As will be appreciated by one skilled in the art, this represents a significant improvement over the prior art as it allows for example a chemotherapeutic agent to be delivered to the brain while keeping the blood brain barrier open only for the period necessary to deliver the agent to the brain.

As discussed herein, ‘an effective amount’ refers to the amount of LPA needed to open the blood brain barrier for the desired period of time and/or the amount of S1P required to close the blood brain barrier. As discussed above, the duration of time that the blood brain barrier is open is somewhat dependent on the dosage administered and accordingly can be determined by one of skill in the art through routine experimentation using methods described herein. In other embodiments, an effective amount of LPA may be between 0.1-5.0 mg/kg or 0.1-4.0 mg/kg or 0.1-3.0 mg/kg or 0.1-2.0 mg/kg or 0.1-1.5 mg/kg or 0.1-1.0 mg/kg subject.

In other embodiments, a sustained iv. Infusion of LPA is used to control the magnitude of BBB disruption. In these embodiments, lower doses of LPA, for example, 0.01-0.1 mg/kg subject is administered as a continuous intracarotid artery infusion, thereby controlling the magnitude of permeability increases by the time of infusion.

As discussed below, S1P causes a rapid and reversible decrease in blood-barrier permeability. Accordingly, S1P can be co-administered to an individual in need of such treatment for decreasing brain blood-barrier permeability. While it is understood that the temporal and reversible opening of the blood-brain barrier is advantageous for drug delivery, there are also pathophysiological conditions where blood-brain barrier disruption for extended periods of time can be detrimental. Therefore, in some embodiments, S1P may be administered to an individual who has suffered neurological trauma or other similar insult including although by no means limited to stroke, multiple sclerosis and the like. In other embodiments, S1P is co-administered with a neural degenerative agent, for example, a pharmaceutical agent, a gene therapy agent or the like. Examples of such neural therapy agents are well known to those skilled in the art. Specifically, in these embodiments, S1P is coadministered with an agent that in the absence of S1P would cross the blood-brain barrier and have deleterious effects.

In other embodiments, S1P is administered to an individual in need of such treatment, that is, an individual who has been administered an agent known to open the blood brain barrier, for example, the osmotic or Cereport mediated methods known in the prior art. Accordingly, in another aspect of the invention, there is provided the use of S1P to counteract a disrupted blood brain barrier due to pathophysiological conditions, for example, stroke, MS, brain trauma or the like, or pharmacological treatments, for example, but by no means limited to osmotic disruption, Cereport and the like. In other embodiments of the invention, there is provided a method of counteracting a disrupted blood brain barrier in an individual in need of such treatment comprising administering to said individual an effective amount of sphingosine 1-phosphate (S1P). It is to be understood that the S1P counteracts the disrupted blood brain barrier in that S1P reduces the permeability of the blood brain barrier compared to an untreated control of similar condition.

Similar to LPA, it is believed that S1P is metabolized by sphingosine lyase and sphingosine phosphotases, and accordingly both S1P and LPA have fairly short half lifes, in the order of minutes.

It is further noted that the effects of S1P are dose dependent and the effects on permeability occur quickly and dissipate quickly with both happening on the order of minutes.

As discussed above, the duration of time that the blood brain barrier is closed and the rapidity with which this occurs is somewhat dependent on the dosage of S1P administered and accordingly can be determined by one of skill in the art through routine experimentation using methods described herein. In other embodiments, an effective amount of S1P may be 0.01-1.0 mg/kg, 0.05-1.0 mg/kg or 0.1-1.0 mg/kg.

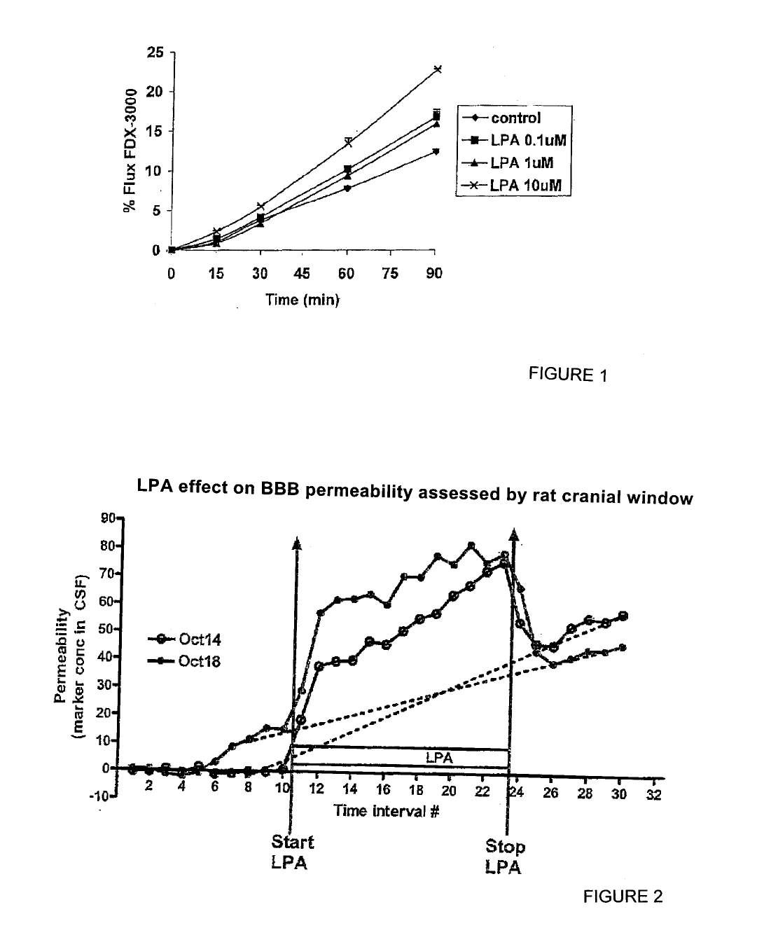

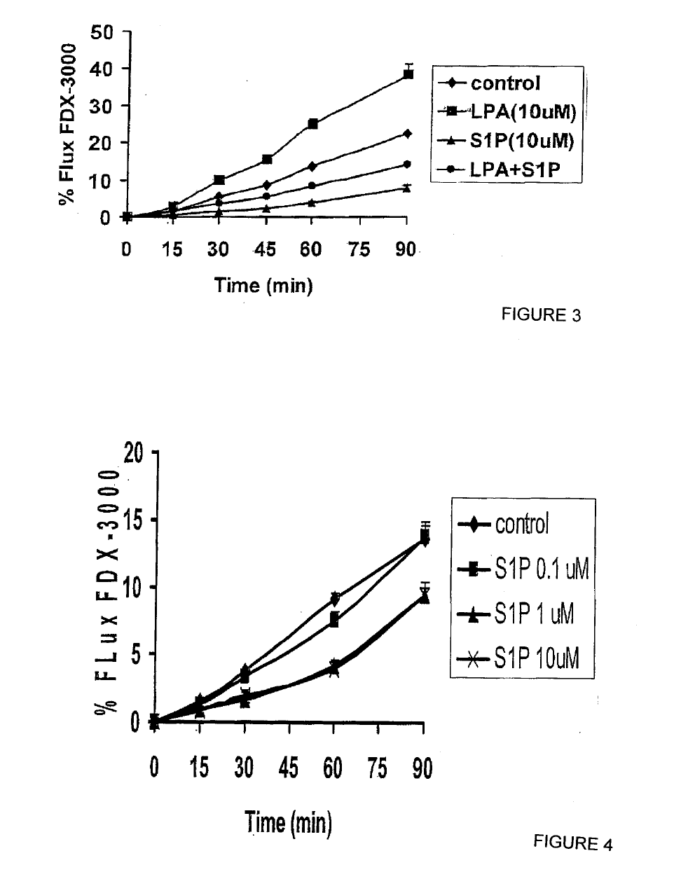

LPA causes a rapid and reversible increase in blood-brain barrier permeability. We have demonstrated this in vitro using both primary cultured bovine brain microvessel endothelial cells (see FIG. 1) and in situ studies using the rat cranial window model, where LPA is applied directly to the cerebral microvasculature (see FIG. 2). It is important to note that the LPA effects are concentration dependent and have onset and reversal in the order of minutes. In addition, we have observed that in the cell culture model of the blood-brain barrier, the increases in permeability produced by LPA can be totally prevented when applied with the phospholipid, S1P (see FIG. 3). The ability of S1P to prevent the permeability increases observed with LPA are important as it provides yet another way to modulate the magnitude and duration of LPA-induced blood-brain barrier disruption. The importance of these observations to the technology is described below. These data also further support the mediation of these effects by specific mechanisms rather than non-specific cell disruption, since the latter mechanism should not be susceptible to counter-action by another lipid mediator.

The delivery of drugs to the brain is limited by the blood-brain barrier. While conventional wisdom holds that large macromolecules such as peptides, proteins, and oligonucleotides are impermeable to the blood-brain barrier, in reality even small drug molecules have problems achieving therapeutically significant levels in the brain. There is currently a great deal of interest in improving drug delivery to the brain. One approach is to transiently open the blood-brain barrier. The best example of this is the use of osmotic agents, such as mannitol, to “shrink” the brain endothelial cells, thereby breaking the tight junction complexes between the cells to allow drug to passively diffuse into the brain via paracellular diffusion. Other examples include the use of bradykinin analogs, like RMP-7/Cereport, to cause temporary disruption of the blood-brain barrier. Currently these agents are used to enhance chemotherapeutic drug delivery to brain tumors. The delivery of anti-HIV drugs to the brain to prevent HIV encephalopathy and AIDS dementia and to destroy this somewhat protected reservoir of HIV would be another potentially important therapeutic application of our approach. Applications of blood-brain barrier disruption as a tool for enhancing gene delivery to the brain for genetic therapy have also been explored. The advantage of our technology over others is that the blood-brain barrier disruption by LPA is much quicker in onset and reversal. This provides a shorter duration of time that the blood-brain barrier is open and thus decreases the chances of toxicity associated with disruption therapy. Secondly, with osmotic and RMP-7 disruption, there is no known way to control the magnitude of the response after the agent is administered. The rapid hydrolysis of LPA together with the ability of S1P to counter the LPA effect by a receptor-mediated (and therefore regulatable) mechanism provides a critical method for additional control of the magnitude and duration of disruption.

The studies show that LPA produces a rapid and substantial increase in brain microvascular permeability as assessed in cultured brain endothelial cells. In the cultured brain endothelial cells, LPA increased cell monolayer permeability when applied to either the apical or basolateral side. This effect was rho kinase mediated. In contrast, S1P caused reductions in brain microvessel permeability, and was able to prevent the permeability increases observed in response to LPA and other permeability enhancing agents. Taken together, these data suggest that LPA and S1P have opposing effects on brain microvascular permeability. S1P also abolished the permeability effect of ethanol and serum.

The current study evaluated the effect of tWOendogenous compounds, LPA and S1P on brain microvessel endothelial cell permeability. In the case of LPA and S1P, the prevailing thought is that LPA increases permeability in brain endothelial cells while S1P decreases permeability in peripheral vasculature (Wiranowska et al., Neurooncol, 14: 225-236, 1992; Fidler et al., Lancet Oncol, 3: 53-57, 2002)

All these agents i.e. LPA and S1P are GPCR mediators (Wolff et al., Klin Padiatr, 209, 275-277, 1997; Rice et al., J Mol Neurosci, 20: 339-343, 2003). Our studies indicate that these GPCR mediators affect the BBB integrity. Hence these GPCR modulators can be used for drug delivery to the brain. They are quick and the BBB can be better regulated with the use of these GPCR modulators than any other currently used technique (Feltner et al., J Clin Invest, 110: 1309-1318, 2002; Henson et al., J Neurooncol, 14: 37-43, 1992)

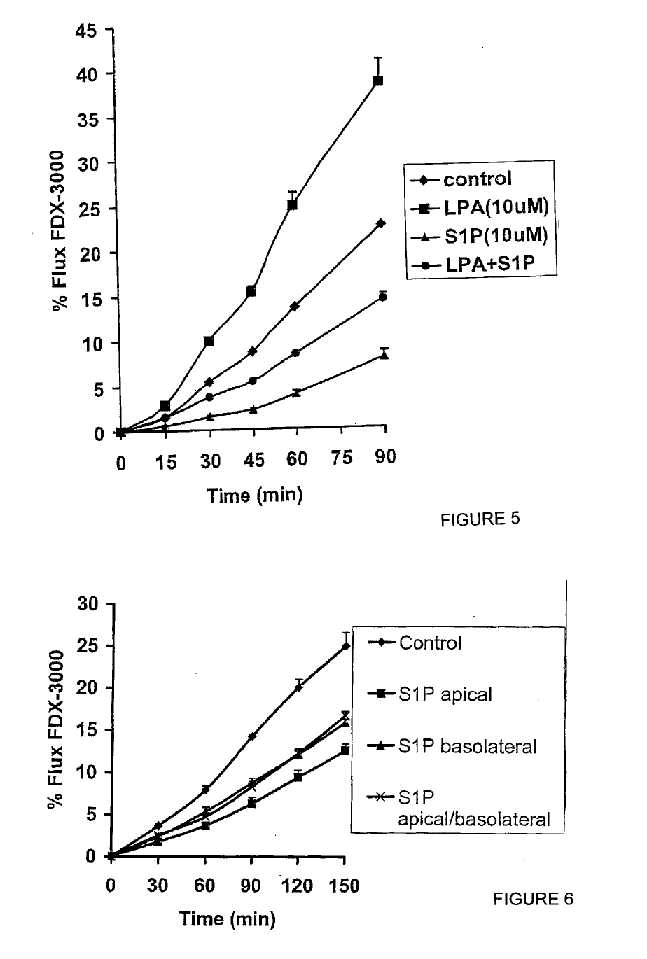

In the current study LPA and S1P produced rapid and significant changes in brain microvessel endothelial cell permeability. Both LPA and S1P showed a concentration dependent effect on brain microvessel endothelial cell permeability. Exposure to LPA caused as much as a two-fold increase in permeability in brain microvessel endothelial cells, while exposure to S1P produced an equally as robust decrease in permeability. There were no polarity issues associated with LPA or S1P (FIGS. 5-7).

The increase in the permeability of brain endothelial cells by LPA corroborates with the study reported by Staddon et. al (Schulze et al., J Neurochem 1997 March 68(3):991-1000) and Galla et. al. (Nitz et al., Brain Res 2003 Aug. 15; 981(1-2)30-40). However LPA's effect in the peripheral vasculature is varied. Studies by Morton et. al. indicate that LPA might be involved in decreasing the permeability of bovine pulmonary artery (Minnear et al., Am J Physiol Lung Cell Mol Physiol 2001 December; 281(6):L1337-44). Similar observations were reported by Haselton et. al. (Alexander et al., Am J Physiol 1998 January; 274(1 Pt 2):H115-22).

S1P cause a decrease in the permeability of peripheral endothelial cells. Permeability effect of S1P in brain endothelial cells is similar to their effects reported in the peripheral vasculature. Garcia et. al., reported that S1P decrease the endothelial permeability in human pulmonary artery endothelial cell (McVerry and Garcia, Cell Signal 2005 February; 17(2):131-9). They also reported that decreases in lung permeability with S1P is due to S1P1 receptor. Their studies suggest that S1P-induced recruitment of S1P1 to caveolin-enriched microdomains fractions promotes PI3kinase-mediated Tiaml/Racl activation required for alpha-actinin-1/4-regulated cortical actin rearrangement and EC barrier enhancement (Singleton et al., Faseb J 2005 October; 19(12):I 646-56).

Apart from the permeability effect on the brain vasculature S1P is also involved in cell proliferation, cell migration, vasoconstriction and prevents apoptosis in cerebral endothelial cells (Waeber et al., Drug News Perspect 2004 July-August; l7(6):365-82). S1P has antiangiogenic role in brain vasculature, however it has a proangiogenic effect on peripheral vasculature (Pilorget et al., J Cereb Blood Flow Metab 2005 September; 25(9):1 171-82), while LPA affects endothelial cell cytoskeleton, proliferation, cell-cell adhesion molecule expression, and vascular permeability (Panetti TS. Biophys Acta 2002 May 23; 1582(1-3):190-6). LPA weaken the barrier properties of cultured porcine brain capillary endothelial cells in vitro (Nitz et al., 2003; Schulze et al., 1997). Our studies supported this report. S1P decreased the permeability of FDX-3000 across the bovine brain microvessel endothelial cells. These compounds had a direct effect via their receptors expressed on the cerebral vasculature. We are the first group to report the permeability effects of S1P on the BBMEC to the best of our knowledge.

S1P, when added in combination with LPA, abolished the permeability increase effect of LPA (FIG. 5). This effect of S1P can be exploited to better regulate the LPA permeability increase effect on the brain vasculature. LPA and S1P can be delivered in such a way that BBB is opened transiently by LPA which would allow transport of drug to the brain. This can be followed by S1P action which will bring the BBB integrity back to normal. Thus use of LPA and S1P together will help in increasing the distribution of drug to the brain and give a better control over its barrier modulation.

Our in vivo studies show that LPA produces a rapid and substantial, increase in brain microvascular permeability as assessed in both the in vivo experiments and the rat cranial window model (FIG. 5). In both preparations, LPA had a quick onset of action having immediate changes within 15 minutes in the mouse. The magnitude of the permeability increase observed in the BBB following LPA treatment is substantial (almost 20-fold). Equally important is the rapid reversal of BBB permeability observed following cessation of LPA infusion in the cranial window model, thus indicating that LPA effect is quick and more importantly reversible. This is the first demonstration of increase of BBB permeability with LPA in vivo to the best of our knowledge. This corroborates with our in vitro results as discussed above. In BBMEC monolayer LPA had a quick and almost 2-fold increase in the permeability when added on the luminal side. Thus the in vivo studies have proved the permeability increase effect of LPA in the BBB.

### Determination of LPA and S1P Effects on Endothelial Cell Monolayers:

Permeability studies were performed on both primary cultured bovine and porcine BMEC permeability. LPA produced a concentration-dependent increase in bovine and porcine BMEC permeability (FIGS. 1 and 3). The lower LPA concentrations (0.1 and 1 uM), produced an almost 1.5 fold increase in monolayer permeability, while the highest concentration (10 uM) resulted in a 2-fold increase in the permeability of BBMEC monolayers. In both brain inicrovessel preparations, the onset of LPA's actions occurred as early as 15 minutes following exposure to LPA and continued for the duration of the experiment.

In contrast, treatment of BBMEC with S1P resulted in concentration-dependent decreases in permeability (FIG. 4). The lowest concentration of S1P examined, (0.1 uM), had no significant effect on the BBMEC permeability however higher concentrations such as 1 and 10 uM caused a 2-fold decrease in permeability of BBMEC. As observed with LPA, the onset of the permeability effect was as early as 15 minutes and lasted for 90 minutes in the presence of S1P.

### Permeability Effect of LPA and S1P Combination:

Given the permeability reducing properties of S1P, studies were performed to examine whether S1P could counteract the permeability effects of various enhancing agents. Exposure of LPA to BBMEC monolayers produced a 2-fold increase in permeability. However, when co-administered with S1P (10 uM), the permeability response to LPA was abolished (FIG. 5).

### Polarity Studies:

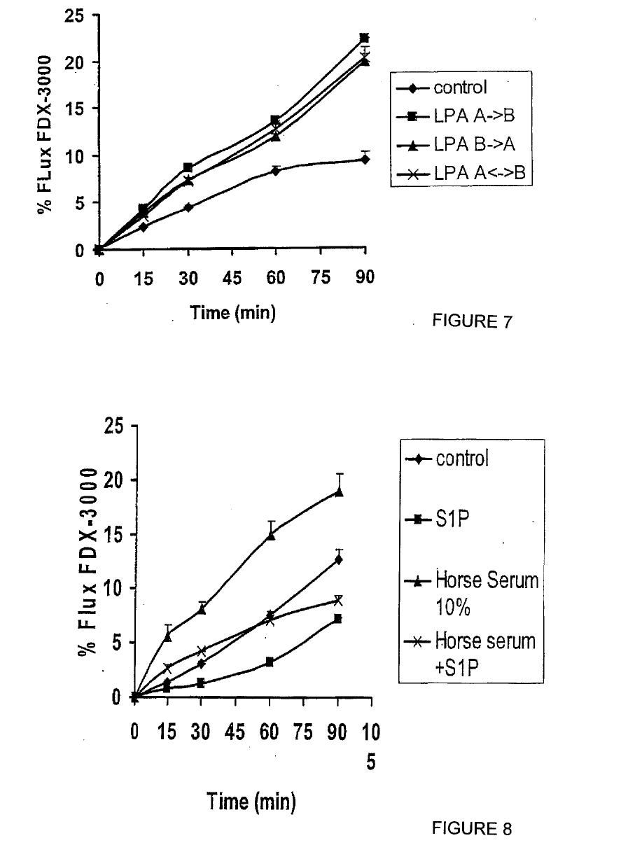

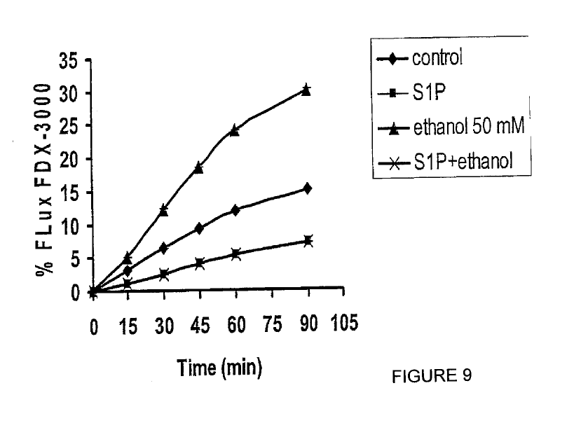

To determine if the site of application of LPA and S1P had an effect of permeability responses in brain endothelial cells, LPA (10 uM) and S1P (10 uM) were applied to apical and/or basolateral side of the monolayer. Exposure of LPA (I 0) on either the apical and/or basolateral side of BBMEC monolayers produced similar increases in monolayer permeability. There was a 2-fold increase in permeability following either apical or basolateral application of LPA (10 μM) to BBMEC monolayers (FIGS. 5 and 7). While S1P decreased BBMEC monolayer permeability (approximately 2-fold), responses to S1P were similar whether applied apically or basolaterally (FIGS. 5 and 6). These studies are important as they indicate that BBB modulation can be achieved through activation of LPA and/or S1P receptors from either the luminal (blood) or basolateral (brain) side.

LPA leads to actin stress fiber formation, contraction of endothelial cells and intercellular gap formation thereby increasing endothelial permeability. The permeability effect of LPA is mediated by the activation of Rho kinase. This enzyme phosphorylates and inhibits the myosin light chain (MLC) phosphatase thereby increasing the phosphorylation of C. Phosphorylated myosin interacts with actin filaments and causes cell contraction. Lysophosphatidic acid has been shown to induce Rho kinase-mediated inhibition of MLC phosphatase and increase MLC-phosphorylation in human endothelial cells independent of activation of Cat+-dependent MLC kinase. Activation of Rho and Rho kinase also mediates the disassembly of adherens and tight junctions in endothelial cells, which leads to a loosening of the tight junctions. Loose tight junctions cause increase in the paracellular permeability of molecules across the monolayer. In our studies we observed an increase in the paracellular permeability of FDX-3000 across the BBMEC monolayer in presence of LPA. However when the rho kinase inhibitor, Toxin B, was added along with LPA there were no significant permeability changes in the BBMEC monolayer permeability. Hence, our studies indicate that LPA acts through rho kinase pathway in cerebral endothelial cells too (FIG. 4).

Another objective of the current study was to determine whether the effects of S1P were selective for LPA or whether it could also prevent permeability increases observed to various stimuli. To address this question a variety of permeability enhancers were examined including serum, C5a, and ethanol (FIG. 8). The present studies suggest that the ability to prevent permeability increases is a generalized function of S1P in brain endothelial cells. As LPA is a serum factor, it could be argued that a portion of the permeability increases observed with serum are attributable to LPA. However, ethanol is reported to directly activate a myosin light chain kinase (NECK) pathway. The fact that S1P also prevents the permeability increases observed with ethanol suggests that activation of S1P receptors can influence permeability to a wide range of compounds. Studies by Garcia et. al. suggest that the direct binding of MLCK to cytoskeletal proteins cortactin is essential in mediating lung vascular barrier augmentation evoked by S1P. This suggests that S1P effects might be useful for inhibiting the permeability increase effect of other permeability enhancers.

All the in vitro studies using LPA and S1P suggest that the rapid hydrolysis of LPA together with the ability of S1P to counter the LPA effect by a receptor-mediated (regulated) mechanism provides a critical method for additional control of the magnitude and duration of disruption. This is not possible with pre-existing methods to open the BBB such as, osmotic disruption or RMP-7 (a bradykinin analog). Thus these phospholipids may have important application in the delivery of drugs to the brain.

LPA and S1P can be used as potential reversible short term modulators of BBB permeability. They are fast acting, reversible, activate from the blood side and can be administered with an agent of interest, as discussed above. Thus these agents can be good cerebral permeability modulators and can help to increase the distribution of drugs to the brain.

Taken together, these data suggest that phospholipid receptors can be used as targets for enhancing drug permeability to brain. LPA and S1P can be used in conjunction to transiently modulate blood brain barrier permeability. Based on these studies, it is clear that the delivery of a wide variety of therapeutic, prophylactic or diagnostic agents to the brain could be achieved with LPA. The use of LPA and S1P to modulate BBB permeability has 3 major advantages over current methods for BBB modulation: (1) quick onset of action, (2) acts on whole brain, and not just the blood tumor barrier and, (3) controlled duration of action. Due to these factors there is less chance of toxicity.

### In Vivo Permeability Studies:

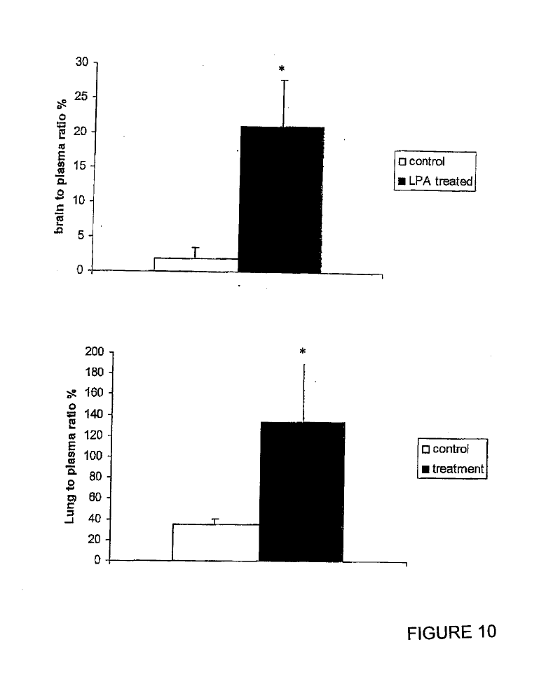

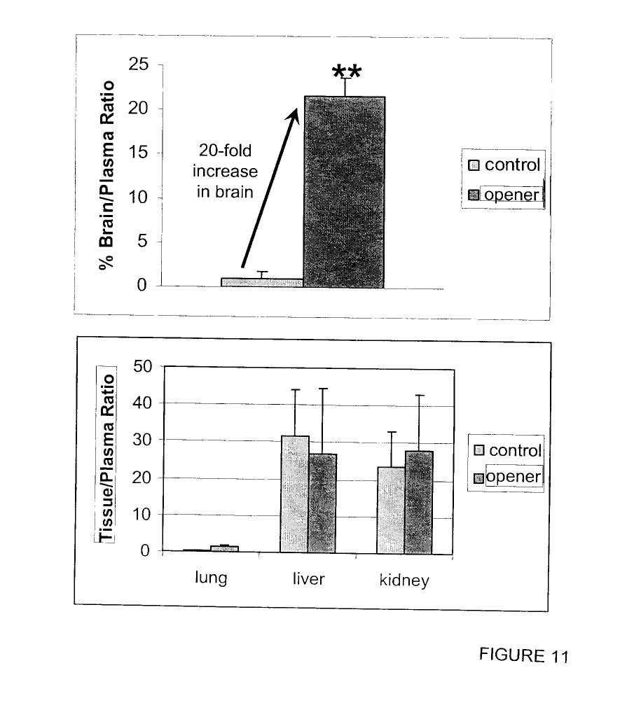

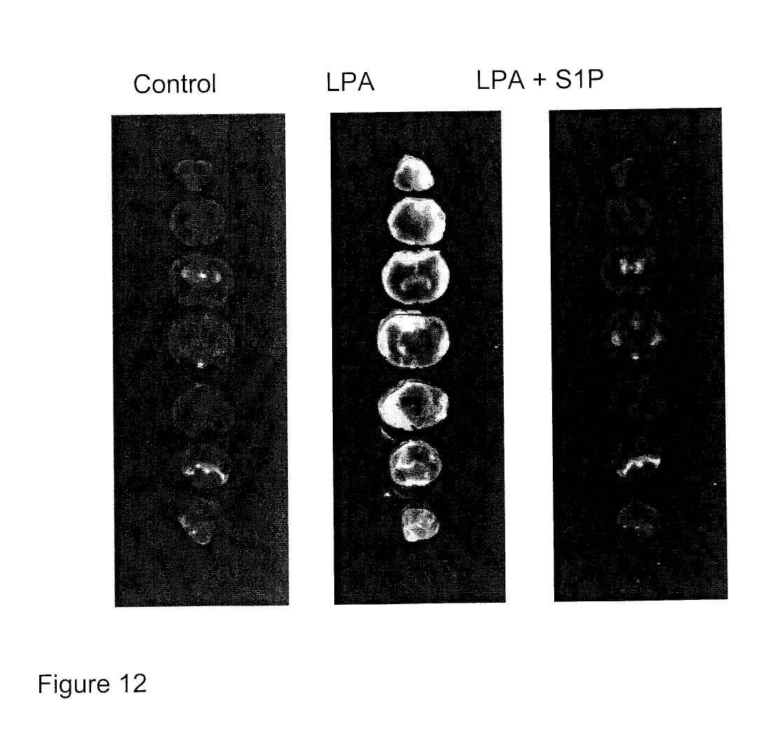

Under normal conditions, the BBB penetration of methotrexate is minimal (FIG. 10). Fifteen minutes after systemic administration, the brain to plasma ratio of ³H-methotrexate in control mice was approximately 2.0%. Treatment of the mice with LPA (1 mg/kg) resulted in significant (approximately 10-fold) enhancement in the brain accumulation of 3H-methotrexate, with brain to plasma ratios for ³H-methotrexate reaching approximately 20% (FIG. 10). Although the magnitude of increase was not nearly as great, systemic LPA injections produced an approximately 3-fold enhancement in 3H-methotrexate accumulation in the lung (FIG. 10). It should be noted that LPA had no effect on the tissue accumulation of ³H-methotrexate in the liver or kidney.

### Effects of S1P on Various Permeability Enhancers:

Ethanol (50 mM) increased BBMEC monolayer permeability by 3-fold while horse serum (10%) showed a 2-fold increase in permeability of BBMEC monolayers (FIG. 8). When the monolayers were treated with ethanol and S1P there was a decrease in permeability of these monolayers indicating that S1P did interfere with permeability effect of ethanol. Likewise when monolayers were treated with horse serum and S1P together, S1P decreased the permeability effect caused by horse serum (FIG. 8).

### Cranial Window Model:

The effect of LPA on cerebral microvascular permeability was also examined using the in situ cranial window model. In these studies, rats were given i.v. infusion of the permeability marker molecule (fluorescein labeled dextran) and baseline permeability was established. The rats were then given LPA locally on the brain microvasculature. Treatment with LPA (10 uM) resulted in significant increases, more than 4-fold in fluorescein labeled dextran permeability (FIG. 2). The effects of LPA on cerebral vascular permeability were rapid in onset (within minutes) and equally rapid in returning to baseline permeability once LPA administration was stopped. (FIG. 2).

### Materials and Methods

### Isolation and Culturing of Microvessel Endothelial Cells:

The permeability effects of LPA and S1P were examined in various microvessel endothelial cell preparations. Primary bovine and porcine brain microvessel endothelial cells (BBMEC and PBMEC, respectively) were collected from the gray matter of fresh cow and pig cerebral cortices using a combination of enzymatic digestion and centrifugal separation methods previously described. The BBMECs and the PBMEC were seeded (50,000 cells/cm2) on collagen coated, fibronectin treated, 6-well Transwell polycarbonate membrane inserts (0.4 um pore/24 mm diameter). The culture media consisted of: 45% minimum essential medium eagle (MEM), 45% Ham's P 12 nutrient mix, 10 mM HEPES, 13 mM sodium bicarbonate, 50 ug/ml gentamicin, 10% equine serum, 2.5 ug/ml amphotericin B, and 100 ug/ml heparin. The cell cultures were grown in a humidified 37° C. incubator with 5% CO2. The media was replaced every other day, and the BBMEC and PBMEC monolayers were used after reaching confluency (approximately 11-14 days).

Studies were also performed with human brain microvessel endothelial cells (HBMEC). HBMEC were purchased from Cell Systems (Seattle, Wash., USA). Briefly, HBMEC were seeded on to 6-well inserts coated with attachment factor (cell systems) and cultured in CS-C complete media according to manufacturer's information. Culture media was changed every other day and the confluent cells were subcultured at 1:2 split ratio. HBMEC were subcultured with Passage Reagent Group Solutions (Cell Systems). The HBMEC were expanded through two passages before being used in permeability studies at passage number four.

### LPA and S1P Preparation:

Lyophilized LPA and S1P was purchased as lyophilized powder and was reconstituted into a 10 mM stock solution using 0.25% fatty acid-free bovine serum albumin (BSA) in assay buffer and kept at −20° C. until used. Working solutions of the phospholipids and C5a were prepared by diluting in assay buffer (0.25% BSA in the assay buffer).

### Determination of LPA and S1P Effects on Endothelial Cell Monolayers:

Permeability studies were performed on confluent BBMEC and PBMEC, monolayers at 370 C. All the cells were pretreated with assay buffer for 30 minutes at 37° C. The control group was treated with assay buffer (0.25% BSA [fatty acid free]). For these studies, 1.5 ml of assay buffer was added to the lumina] (apical) side of the monolayer and 2.5 ml of assay buffer was added to the ablumenal (basolateral) side of the monolayer. Treatment groups received either LPA (0.1, 1, and 10 uM) or S1P (0.1, 1, 10 uM), alone and in combination, on both sides of the inserts. Permeability across the cell monolayers was determined by adding 10 uM fluorescein-labeled dextran (FDX-3000; MW 3000) to the donor (apical) compartment. Samples (100 ul) were removed from the receiver (basolateral) compartment at various times (0, 15, 30, 60 and 90 minutes). Samples (100 ul) were also taken from the donor compartment at the start (time 0) and conclusion (90 min) of the experiment. The concentration of FDX-3000 in the donor and receiver samples was measured spectrofluorometrically using a Shimadzu RFU5000 spectrofluorometer (Shimadzu Scientific Instruments, Columbia, Md., USA) with Ex (7 v) at 488 nm and Em (2 k.) at 510 nm. Permeability was expressed as the percent transfer of the fluorescent marker across the monolayers. The percent transfer was determined by dividing the cumulative concentration in the receiver compartment by the original concentration in the donor compartment at time zero. Permeability coefficients were calculated from the following formula:

P=(V/SA*Cd)*Cr/T

V=volume of receiver chamber
SA=surface area of cell monolayer
Cd=conc of marker in the donor at time 0
Cr=the cumulative conc of marker in the receiver at the sample at time T

### Polarity Studies

For studies examining the functional polarity of LPA and S1P (10 4M) of the phospholipids were applied to either the apical or basolateral side of confluent brain microvessel endothelial cell monolayers. There was 30 min AB pretreatment followed by treatment with either LPA and S1P on either apical and/or basolateral side and the flux of FDX-3000 across the monolayer was measured. Permeability across the cell monolayers was determined by adding 10 uM fluorescein-labeled dextran (FDX-3000; MW 3000) to the donor compartment. Samples were removed from the receiver compartment after 0, 15, 30, 45, 60, and 90 minutes. Samples were taken from the donor compartment at the start (time 0) and conclusion (90 min) of the experiment. Samples were analyzed as described above.

### In Vivo Permeability Studies:

Female Balb/c mice were used to assess BBB permeability in vivo. Mice received a bolus injection (ml/kg) of ³H-methotrexate (4 uCi total activity) via the tail vein. The radiolabeled methotrexate was given in combination with either LPA (1 mg/kg) or vehicle (0.25% bovine serum albumin in phosphate buffered saline solution). Fifteen minutes after the injection of methotrexate, mice were anesthetized using intraperitoneal injection of ketamine and xylazine and a sample of blood was removed by cardiac puncture. Following the blood sample, mice were sacrificed and various tissues including, the brain, lung, liver and kidney, were removed for determination of 3H-methotrexate. Blood samples were spun for 8-10 minutes and 100 uL of plasma was collected. Tissue samples were weighed and homogenized with double the volume of tissue solubilizing agent (Scintigest™, Fisher Scientific). A 200 uL aliquot of the tissue homogenate was used to analyse the tissue samples. The samples were then mixed with Ultima Gold™ (Perkin Elmer) scintillation cocktail and analysed by liquid scintillation counter (Perkin Elmer) to determine the amount of ³H-methotrexate present in the blood and tissue. The distribution of ³H-methotrexate was based on the ratio of the radioactivity in the tissue versus the radioactivity in the plasma sample.

### Cranial Window Model:

Cerebral vascular permeability was also evaluated using the rat cranial window model. For these studies male Wistar-Furth rats (n=15) were anesthetized (Inactin; thiobutabarbital 100 mg/kg ip), and a tracheotomy was performed. The rats were mechanically ventilated with room air and supplemental oxygen. A catheter was placed in the femoral artery for the measurement of systemic blood pressure and to obtain blood samples. A catheter was placed in the femoral vein for injection of intravascular tracer FDX 10 10a. To visualize the cerebral microcirculation, a cranial window was prepared over the parietal cortex using methods previously described (Mayhan and Heistad, Am J. Physiol, 248: H712-H718, 1985; Mayhan, Brain Research, 927: 144-152, 2002). An incision was made in the skin and retracted with sutures to expose the skull and serve as a well for the suffusion fluid. Inlet and outlet ports were made in the skin to allow for the constant flow of the suffusate across the cerebral (pial) microcirculation. Finally, a craniotomy was performed, the dura was incised, and the cerebral microcirculation was exposed. The suffusion fluid (artificial cerebrospinal fluid) was heated (37+1° C.) and bubbled continuously with 95% nitrogen and 5% carbon dioxide to maintain gases with in normal limits. Blood gases were also monitored and maintained within normal limits. At the end of the experimental duration, the anesthetized rats were sacrificed with an injection of saturated potassium chloride.

Permeability of the BBB was evaluated by calculating the clearance of FDX 10 kDa (10⁻⁶ ml/s) from the area of parietal cortex exposed by the craniotomy, as previously described (Mayhan and Heistad, Am J. Physiol, 248: H712-H718, 1985; Mayhan, Brain Research, 927: 144-152, 2002). Briefly, the suffusate fluid was collected in glass test tubes with the aide of a fraction collector, and the concentration of FDX in the suffusate fluid during topical application of vehicle (saline) or LPA (10 μM) was determined using a spectrofluorometer. FDX 10 kDa was infused intravenously (40 mg/ml at 0.06 ml/min), and arterial blood samples (60 0sample) were drawn at various time points throughout the experimental time period, and the concentration of FDX 10 1cDa in the blood sample was determined spectrofluorometer (Perkin-Elmer model LS30). A standard curve was prepared on a weight-to-volume basis. The clearance of 1010a FDX was calculated by multiplying the suffusate-to-plasma concentration by the suffusate flow rate (Mayhan and Heistad, Am J. Physiol, 248: H712-H718, 1985; Mayhan, Brain Research, 927: 144-152, 2002). Prior to surgery, animals were anesthetized with an i.m. injection of ketamine mixture (87 mg/kg ketamine 113 mg/kg xylazine). Body temperature was maintained at approximately 37° C. by a heating mat. Animals were placed on a small animal stereotaxic frame. All surgical procedures were carried out under aseptic conditions. The scalp and underlying soft tissue over the parietal cortex were removed bilaterally. A low-speed dental drill with irrigation of artificial cerebrospinal fluid (ACSF) was used to create a rectangular cranial window that extended from the bregma to the lambdoid sutures and was centered on the midsagittal suture. The dura mater was punctured and excised. Finally, a glass plate was sealed to the bone surrounding the cranial window. Care was taken to avoid any unnecessary contact with the brain tissue. After recovery from anesthesia, animals with windows were returned to the animal facilities and were given I week to recover from surgery.

While the preferred embodiments of the invention have been described above, it will be recognized and understood that various modifications may be made therein, and the appended claims are intended to cover all such modifications which may fall within the spirit and scope of the invention.

1. A method of modulating permeability of the blood brain barrier comprising:
administering to an individual in need of such treatment an effective amount of lysophosphatidic acid (LPA) or sphingosine 1-phosphate (S1P).

2. A method of administering a neural agent to an individual in need of such treatment comprising coadministering an effective amount of the neural agent and an effective amount of lysophosphatidic acid (LPA) to said individual; and following a therapeutically suitable interval, administering an effective amount of sphingosine 1-phosphate (S1P) to said individual.

3. A method of counteracting a disrupted blood brain barrier in an individual in need of such treatment comprising administering to said individual an effective amount of sphingosine 1-phosphate (S1P).
